(19) United States
(12) Patent Application Publication
(10) Pub. No.: US 2021/0166794 A1
(43) Pub. Date: Jun. 3, 2021

(54) Genetic Based Health Management Systems for Weight and Nutrition Control

(71) Applicant: Pathway Genomics Corporation, San Diego, CA (US)

(72) Inventors: Michael Nova, Del Mar, CA (US); Andria Del Tredic, San Diego, CA (US); Aditi Chawla, San Diego, CA (US); Victoria Mognuson, San Diego, CA (US)

(21) Appl. No.: 17/174,092

(22) Filed: Feb. 11, 2021

Related U.S. Application Data
(63) Continuation of application No. 15/146,756, filed on May 4, 2016, now abandoned.
Application No. 15/146,756 is a continuation of application No. 13/316,924, filed on Dec. 12, 2011, now abandoned.
Application No. 13/316,924 is a continuation-in-part of application No. 12/804,363, filed on Jul. 19, 2010, now abandoned.

(51) Int. Cl. G16H 10/60; G16H 50/30; G16H 20/60; G16H 15/00; G16H 50/20

(52) U.S. Cl. CPC G16H 10/60; G16H 50/30; G16H 50/20; G16H 15/00; G16H 20/60

(57) ABSTRACT

Systems have been devised for health management based upon genetic markers. Specifically, systems are arranged to compute genetic risk for several factors relating to metabolism and weight gain in view of various genotypes at particular markers in an individual's genome. An algorithm which depends upon these risk calculations and in further view of the presence of additional variants in the genome under test is executed to arrive at a diet type selection for the particular user. In additions to diet type selections, specific supporting diet related recommendations related to eating behaviors, food reactions and nutritional needs, based upon markers found in the genetic profile, are additionally included in a diet action plan.

### CROSS-REFERENCE TO RELATED APPLICATIONS

The present application is a continuation of U.S. patent application Ser. No. 15/146,756. Filed May 4, 2016, which is a continuation of U.S. application Ser. No. 13/316,924, filed on Dec. 12, 2011, which is a continuation-in-part of U.S. patent application Ser. No. 12/804,363, filed on Jul. 19, 2010. The contents of these related applications are hereby incorporated by reference in their entireties.

### BACKGROUND OF THE INVENTION

### Field

The following invention disclosure is generally concerned with genetic health management systems and more specifically concerned with automated systems for providing genome specific action plans for health management as it relates to diet, nutrition and exercise. This application continues in-part from earlier filed patent application having Ser. No. 12/804,363 filed Jul. 18, 2010.

### Prior Art

In view of serious and widespread health crisis, great attention is now directed towards mechanisms effecting human obesity. Indeed, health practitioners from a great many disciplines all work diligently to arrive at new solutions for controlling weight gain. In particular, interesting advance has been realized in diet and genetics fields and various systems and methods therein continue to produce attractive results.

Ready availability and low cost of genetics testing and techniques put DNA based solutions to obesity at the forefront. Each day, researchers find yet additional pieces to this complex puzzle and gain a greater understanding of the overall picture.

Specifically, correlations between health traits, disease, predisposition, lifestyle and diet all may be connected to a person's specific genetic makeup. Polymorphisms and other genetic features in the genetic code are sometimes responsible for metabolic performance including dietary, nutritional and exercise response.

Studies in genetics have suggested that persons having particular genetic compositions may improve their chances in avoiding disease such as obesity and type 2 diabetes by taking certain lifestyle actions—i.e., those relating to diet, nutrition and exercise.

However, these studies are quite complex and as such not readily usable by the general public. Even where skilled practitioners of medicine have access to this information, time constraints among other factors greatly limits its application and practical use. Further, even where such studies could be reduced to practical discrete terms, it has been heretofore quite expensive if not impossible to discover the details of one's personal genetic makeup. Genetic testing has not heretofore been available to those physicians and patients who seek to improve health and more specifically those who seek solutions to weight management. Only those persons so highly motivated and educated could read genetic studies related to obesity, further examine their personal genetic profile for the presence of particular genetic features and markers, and determine a course of action based on that review of the art with respect to their own personal genome.

It is therefore highly desirable to have a machine and system by which a patient merely submit a DNA or other genetic sample, and receives in return and easy-to-use visually driven recommendation package to provide suggestions regarding diet nutrition and exercise most suitable for a particular genetic composition and individual.

One particular invention of significant importance is presented by inventors Bender et al, titled “Genetic Marker Weight Management”. In this teaching of system and method for facilitating personal weight management based on genetic markers—wellness information and macronutrient requirement information may be presented to a user. The system is deployed about a computer network to enable users access via the Internet for example. Details may be learned via US patent application publication 2010/0098809 published Apr. 22, 2010.

Inventors Draper, et al, present another invention of significant importance titled “Genetic markers for weight management and methods of use thereof”. They teach a method and tests for personalized weight loss programs that are based on an individual's genotype at certain metabolic genes. Details may be learned via US patent application publication 2010/0105038 published Apr. 29, 2010.

Further, inventors Gill-Garrison, et al, present an invention titled “Computer-assisted means for assessing lifestyle risk factors”, wherein they teach methods of assessing disease susceptibility by analyzing alleles at genetic loci associated with disease and lifestyle risk factors. The risk is computed and delivered to the consumer via the internet. Details may be learned via U.S. Pat. No. 7,054,758.

Inventors et al. of Massachusetts have identified and patented a human gene relating to obesity. In part, their teaching discloses detection and response to a finding of this gene in a human genome as it relates to weight management. In addition, the invention relates to antibodies to the protein encoded by the discovered nucleic acids. U.S. Pat. No. 7,501,118 contains details.

Renowned genetics research company Myriad Genetics has patented a gene, which relates to obesity and uses of same. Specifically, the invention relates to detection of this ‘obesity’ gene and use in diagnostics of predisposition to obesity and/or diabetes. In U.S. Pat. No. 7,314,713 published Jan. 1, 2008 details will be discovered.

A gene associated with regulation of energy balance is taught in patented invention of U.S. Pat. No. 7,306,920 by Zimmet et al. Filed Jun. 3, 2002 the patent also relates to obesity and diabetes. A protein associated with the modulation of obesity, diabetes and metabolic energy levels is encoded by the claimed gene. The disclosure describes uses of the gene and systems, which might be responsive to the presence of same.

In U.S. Pat. No. 7,302,398 a health management system quantitatively evaluates health using comprehensive indexes of personal health conditions to optimize and advance a healthcare guidance. A predicted period of health life expectancy and related information are displayed by display means or printed out by printing means.

Another obesity gene is discovered, disclosed, described and patented in U.S. Pat. No. 6,998,472 by Robinson et al. The gene used in transgenic animals may induce obesity or infertility.

Rothschild et al, teach in U.S. Pat. No. 6,803,190 issued Oct. 12, 2004 a gene and use of the gene as genetic marker for fat content, weight gain, and feed consumption. The gene being associated with fat content may be useful in selection of animals for breeding.

A gene therapy for obesity invention is presented in U.S. Pat. No. 6,630,346. Inventor Morsy et al describe a gene therapy to treat obesity in animals. The gene delivered to animals encodes leptin or a leptin receptor.

Inventor Brower of California teaches a computerized reward system for encouraging participation in a health management program. U.S. Pat. No. 6,151,586 describes in detail a computer system to assist in health management. The system is distributed over a network or by remote users and may interact with scripts provided by a server to effect a health management program.

In U.S. Pat. No. 5,941,837 a health management and exercise support device are presented. Inventors Amano et al. provide an analysis module, which receives waveform information and body movement information and from analysis of these further provides notifications to interested users.

A system that provides therapy reports for health management is presented as U.S. Pat. No. 5,724,580. A comprehensive management and prognosis report is formed at a centralized data management center for a patient at a remote location. Data from the patient is processed at an analysis module and a report which depends therefrom is formed and transmitted to the user.

While systems and inventions of the art are designed to achieve particular goals and objectives, some of those being no less than remarkable, these inventions of the art have nevertheless include limitations which prevent uses in new ways now possible. Inventions of the art are not used and cannot be used to realize advantages and objectives of the teachings presented here following.

### SUMMARY OF THE INVENTION

Comes now, Michael Nova, Andria Del Tredici, Aditi Chawla and Victoria Magnuson with an invention characterized as genetic based health management systems for weight and nutrition control including both apparatus and methods.

Apparatus and methods are devised to provide action plans relating to health and wellbeing—and more specifically to diet and/or performance and behavior modification. These action plans are highly personalized as they are based in part upon a person's own genetic code. A consumer/user interested in finding a diet or performance plan which cooperates with her own personal genetic composition may submit a biological sample, for example saliva, for processing. A received sample containing genetic material is processed and operated upon to produce a digital genome dataset suitable for processing at a logic processor. Stored logic or code having parameters which depend upon genetic features is run to arrive at an output relating to diet type suggestions. For example, where certain genetic variants are found present in the user's genome, suggestions regarding diets most likely to result in a good response may be proposed. Many studies have now shown that certain genotypes are associated with greater responsivity to one diet or metabolic type, or eating behavior compared to others. As such, when a biological sample is reduced to a form that may be analyzed at a logic processor in view of prescribed well-defined rules, based upon a great body of research, an output may be produced which is useful in guiding a user in selection of diet, metabolic, and nutrition types.

In most important versions, a set of genetic markers is considered to assign a risk value for each of a set of health related conditions. Thereafter, a logic tree is processed in view of these assigned risk values and additionally in further consideration of the presence of other genetic markers in the user's genome. The endpoints of such processing are one of a plurality of diet and nutrition type specifications.

Therefore, a genetics based health management system taught herein may propose weight and nutrition control diet types in addition to other genetics-based diet considerations and suggestions, such as eating and addictive behaviors, based upon an automated genetic analysis.

### Objectives of the Invention

It is a primary object of the invention to provide new genetics based health management systems for diet, performance, and nutrient selection.

It is an object of the invention to provide systems for managing weight, performance, behaviors and nutrition control based upon predicted metabolic response based upon an individual's genetic composition It is a further object to provide automated, easy-to-use, personal health management systems based upon discrete algorithms.

It is an object of the invention to eliminate ‘fuzzy logic’ and variability in results in health management systems, by using genetics through multiple discrete logic paths, which can be executed by a machine.

A better understanding can be had with reference to a detailed description of preferred embodiments and with reference to the appended drawings. Embodiments presented are particular ways to realize the invention and are not inclusive of all ways possible. Therefore, there may exist embodiments that do not deviate from the spirit and scope of this disclosure as set forth by appended claims, but do not appear here as specific examples. It will be appreciated that a great plurality of alternative versions are possible.

### BRIEF DESCRIPTION OF THE DRAWING FIGURES

These and other features, aspects, and advantages of the present inventions will become better understood with regard to the following description, appended claims and drawings where:

FIG. 1 is an overall system block diagram showing most important elements and their relations with the others;

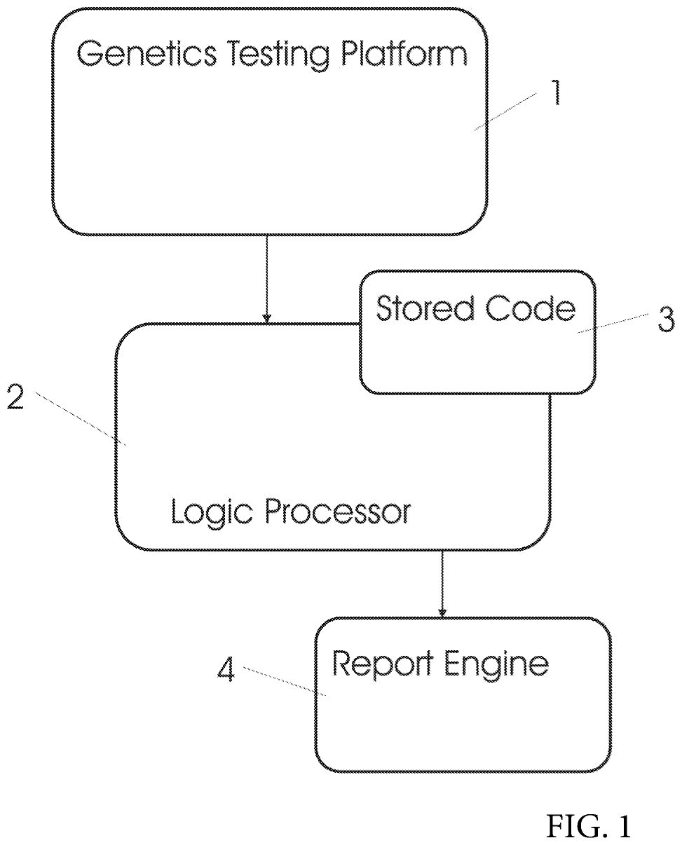

FIG. 2 is a detailed block diagram further defining important elements and relationships;

FIG. 3 is a method block diagram illustrating general steps of these methods;

FIG. 4 is a detailed example logic tree of these systems; and

FIG. 5 is an illustration of a diet action plan and its components.

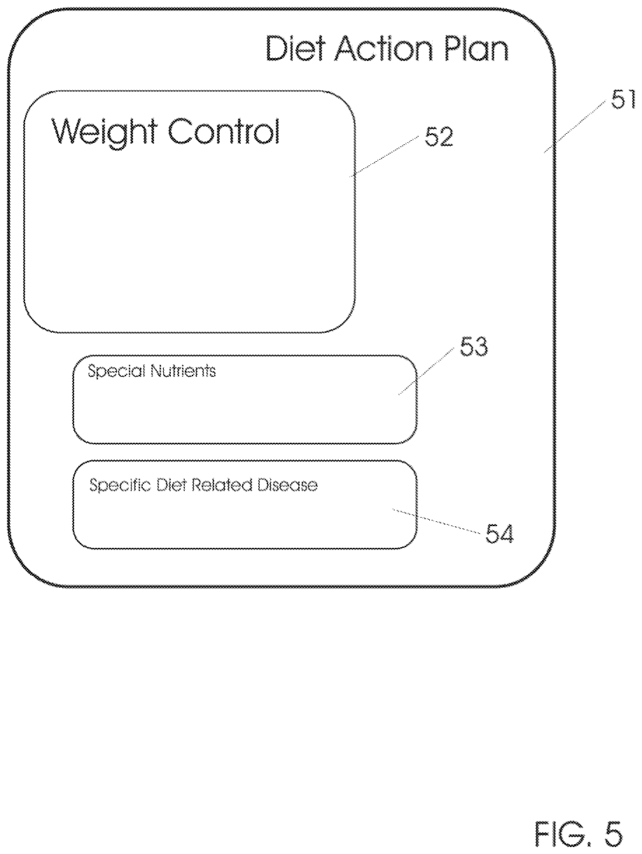

### PREFERRED EMBODIMENTS OF THE INVENTION

In accordance with each of preferred embodiments of the invention, automated health management systems are provided. It will be appreciated that each of the embodiments described include an apparatus and that the apparatus of one preferred embodiment may be different than the apparatus of another embodiment. Accordingly, limitations read in one example should not be carried forward and implicitly assumed to be part of an alternative example.

Genetics based health management systems are presented in which genetic material from a human individual is received at a genetics testing platform 1, purified, amplified, reacted, and scanned to form a digital representation of portions of the test subject's genome or a digital genome dataset. The digital genome dataset which is comprised of discrete values is passed to a logic processor for further processing in accordance with application specific program code which may be run by the logic processor.

Prescribed stored program code includes application code, a plurality of specific logic modules in a rules library, and a risk assignment module. Based upon information from the specific person under test, a risk assignment module assigns discrete risk values for each of a plurality of disease conditions or related attributes. Information from which these risk assignments are based may be purely genetic markers, may alternatively be based upon genetic markers and lifestyle factors as expressed in a survey, or may additionally include family history and other consideration. In all versions, discrete values are assigned to each disease condition. For example, one version of these risk assignment modules is used to assign binary risk values ‘high’/‘low’ for each of the health conditions designated as: decreased HDL cholesterol levels; elevated LDL cholesterol levels; elevated blood sugar (BS), and elevated triglycerides (TG).

In some important versions, a binary value of either ‘high’ or ‘low’ is assigned for every condition described above in view of a user's genetic make-up. In alternative schemes, a ‘high’, ‘medium’, low’ risk assignment is made. Still further in other alternative versions, a risk value scheme includes ‘high’; ‘above average’; ‘average’; ‘below average; and ‘low’. Further alternative versions may contain any number of risk value assignments. It is possible that the scheme may include any number of risk value assignments, and the number that best suits the particular test/disease will be adopted for use. In any system to express risk as a discrete value, a risk factor is assigned to each of these, and the assigned risk factors are used in processing matrices of logic modules of the rules library. The essence of the invention depends upon the nature of the risk calculations not the degree of resolution of risk. It will be considered merely ‘fine-tuning’, that adjustments to the basis from which these risk values are assigned are possible, and vary from in the many versions or implementations of these systems.

In furtherance of the specific example, after risk value assignments to each of HDL, LDL, BS, and TG, a logic module is recalled from the rules library. A logic module has a plurality of parametric inputs coupled to portions of the digital genome dataset and to the risk values assigned in the previous step. The logic processor executes the logic of the particular rule and arrives at a result set which may include information particular to a specified action plan—for example an action plan for diet. A result set may be as simple as a diet type specification, or may include a diet type specification and many additional elements such as specific food references in view of single SNP disease associations as well as special diets suitable for diseases in which there is a substantial genetic contribution such as Celiac disease. Other genetic markers such as those for bitter taste or lactose intolerance or satiety or eating disinhibition, may also be used as a part of the logic module to guide the most suitable nutrition plan for the individual.

These result sets are passed to a report engine having prescribed templates containing dynamic visual elements that can be modified in accordance with values in the result set. The report engine prepares a template by applying values and settings to all of its dynamic elements to arrive at a health report regarding diet type selection as well as additional nutrition related recommendations.

Stored program code 3 includes a rules library of executable code modules and a risk assignment module. The output of the logic processor is communicatively coupled to the report engine 4, which operates to execute templates in view of result sets provided by the logic processor. The report engine. also operates to deliver these reports as completed documents that are highly specific to the user by way of their dependence on the user's genomic features.

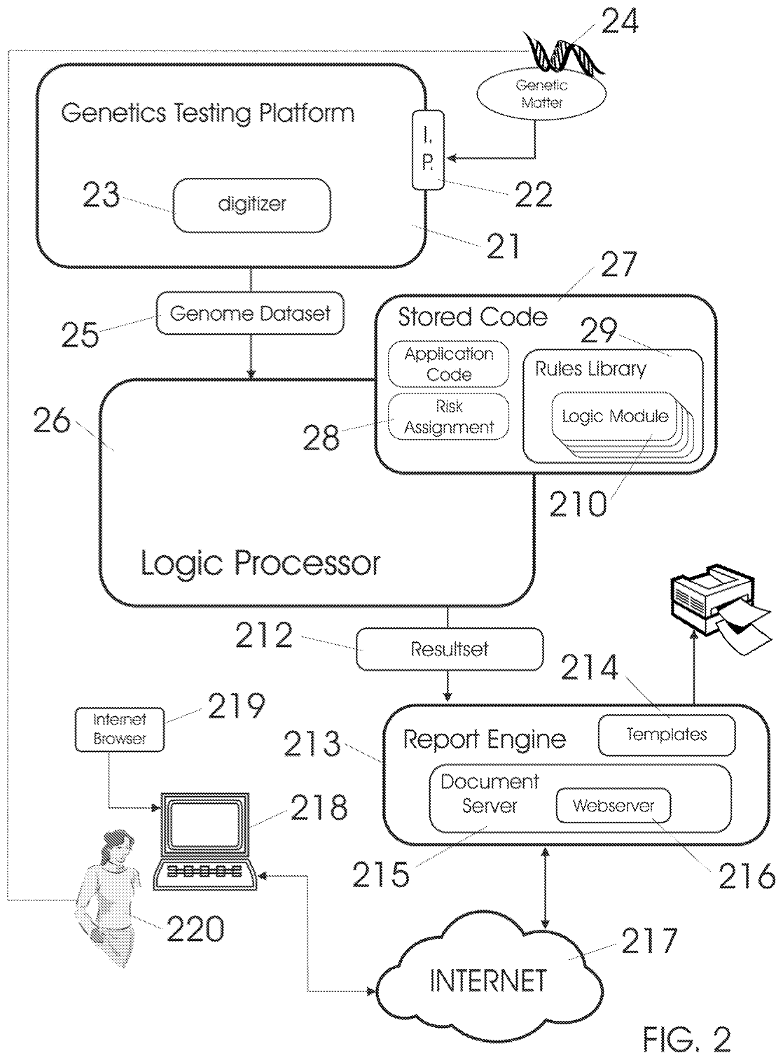

With reference to FIG. 2 which illustrates apparatus of these systems with an increased level of detail, a genetics testing platform 21 having an input port 22 and digitizer 23. A DNA sample 24 from a user is received at the input port, converted into a digital genome dataset 25 and conveyed to the logic processor 26 to which the genetics testing platform is coupled.

Stored program code 27 includes application code for execution of all application functionality, risk assessment module 28 and rules library 29 comprising a plurality of logic modules 210. Normal running of the application code invokes a risk assignment for the individual under test. In view of genetic information contained in the digital genome dataset, a risk value is assigned for each of HDL, LDL, TG, and BS.

After risk values are assigned for each of these, a logic module from the rules library is recalled and executed at the logic processor in view of the assigned risk values. Execution of recalled logic modules lead to an endpoint specification of diet, exercise, performance, metabolism or any other parameter. In addition to a generalized diet type, the logic endpoint may additionally include some additional specific diet recommendations. A diet action plan may additionally include added conditions or exclusions related to health, including those not related to weight control.

The output of these logic modules executed at the logic processor is embodied as a result set 212 of values, which drive report template preparation. A report engine 213 receives a result set from the logic processor to which it is in communication. The report engine is comprised of prescribed document templates 214 relating to diet and nutrition, and a document server 215.

Dynamic visual objects of the template such as text fields, graphs, charts, illustrations, logos, recipe sets, et cetera, are responsive to information contained in the result sets. The report engine document server is arranged to, and is operable for transmitting completed reports to display systems such as common printers and/or computer workstations enabled with Internet browsers. In one import version, dynamic interactive reports are encoded as XML and transmitted by a Webserver 216 over the Internet 217 to a remote workstation 218 having suitable Internet browsing software 219 where it may be displayed and manipulated by an authorized user 220.

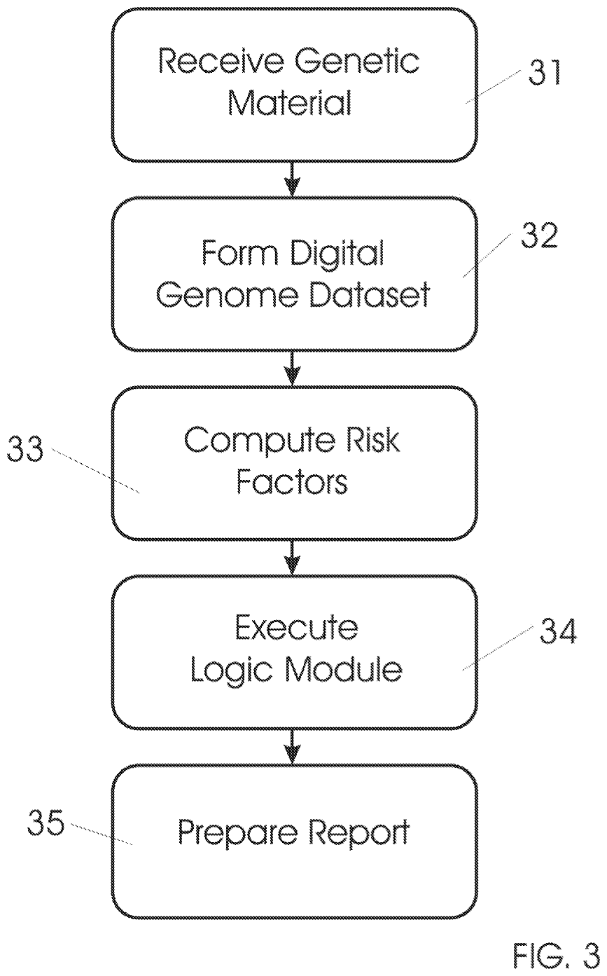

One will appreciate in more detail methods included as part of these systems in view of the drawing FIG. 3.

In a first step, genetic matter is received 31 from a donor person having interest in health maintenance based upon genetics and more specifically genetic based selection of diet, metabolism, and nutrition types. By submitting a saliva sample by mail or in person, a user easily delivers and introduces to the system sufficient genetic material for processing in accordance with these methods.

Received genetic material is purified and amplified, and then reacted in a second method step in which a digital genome dataset is formed. Genetic probes which are specifically chosen with a view to identifying the presence or absence of certain specific genetic features or markers related to diet and metabolism. After reactions with genetic probes, the reactions are illuminated and the optical signals are subjected to a threshold to yield a binary indication of the presence/absence for each genetic marker or feature. Accordingly, after this step, a digital genome dataset is produced and passed on to a logic processor.

In a computational step, step 33, risk factors are computed and assigned for at least four important disease related conditions including: elevated LDL; ‘decreased HDL; elevated triglycerides (TG) and elevated blood sugar (BS). These risk factor calculations can depend upon weighting factors in view of the strength of various risk indicators for each. For each risk calculation, based upon sets of various genetic markers, particular markers will have weights associated therewith to guide overall risk assignment with respect to any of the stated conditions. In some useful versions, risk may be expressed merely as a binary ‘high’/‘low’.

After risk is calculated, a logic module having therein a logic tree is executed 34. A logic module has inputs related to risk and inputs related to features of the genome dataset.

Values from the genome dataset and values from the risk assignments drive and control execution of these logic modules. After a logic module is executed, an output including at least diet type specification (diet type relating to weight and nutrition control) and in some cases additional cooperating diet recommendations, is sent to a report engine where a ‘prepare report’ step 35 is performed. Based upon results from processing as described, a template of the report engine is modified and manipulated whereby dynamic objects therein are set to specific states to reflect the results of the logic module execution.

For illustration purposes, the following example data are provided. Where a person submits to the system and provides a genetic sample, which is converted to a digitized genome dataset. Thereafter, certain SNPs are considered in a risk computation and assignment step.

In a risk assignment for HDL, the following SNPs are considered:

TABLE I

| SNP Marker | Gene |
|---|---|
| rs 1883025 | ABCA1 |
| rs2967605 | ANGPTL4 |
| rs173539 | CETP |
| rs174547 | FADS1 |
| rs4846914 | GALNT2 |
| rs1800961 | HNF4A |
| rs2338104 | KCTD10 |
| rs2271293 | LCAT |
| rs10468017 | LIPC |
| rs4939883 | LIPG |
| rs12678919 | LPL |
| rs7679 | PLTP |
| rs471364 | TTC39B |
| rs964184 | ZNF259 |

Each of these has associated therewith a weighting factor. When any of those are found in the example genome dataset, the weighting factor is applied in an overall calculation of risk. For each of these SNPs a different weighting value may yield variable impact on the overall HDL risk assignment which is finally a binary value either ‘high’ or ‘low’.

In a risk assignment for elevated blood sugar, the following SNPs are considered:

TABLE II

| | |
|---|---|
| rs11708067 | ADCY5 |
| rs10885122 | ADRA2A |
| rs11605924 | CRY2 |
| rs174550 | FADS1 |
| rs560887 | G6PC2 |
| rs4607517 | GCK |
| rs780094 | GCKR |
| rs7034200 | GLIS3 |
| rs11071657 | C2CD4B |
| rs2191349 | Intergenic |
| rs7944584 | MADD |
| rs10830963 | MTNRIB |
| rs340874 | PROX1 |
| rs11920090 | SLC2A2 |
| rs13266634 | SLC30A8 |
| rs7903146 | TCF7L2 |

Again, each of these may be considered with a separate weighting factor to arrive at a final ‘high’ or ‘low’ specification for elevated blood sugar risk in view of actual SNPs present in the dataset.

Similarly, a risk factor for elevated LDL can be determined in consideration of finding or not finding the following SNPs in the specific subject's genome dataset

TABLE III

| | |
|---|---|
| rs6544713 | ABCG8 |
| rs515135 | APOB |
| rs12740374 | CELSR2 |
| rs3846663 | HMGCR |
| rs2650000 | HNFIA |
| rs1501908 | Intergenic |
| rs6511720 | LDLR |
| rs6102059 | MAFB |
| rs.10401969 | NCAN |
| rs11206510 | PCSK9 |
| rs4420638 | APOC1 |

In addition the risk factor for elevated triglycerides (TG) is computed in view of the following SNPs:

TABLE IV

| | |
|---|---|
| rs10889353 | ANGPTL3 |
| rs7557067 | APOB |
| rs174547 | FADS1 |
| rs1260326 | GCKR |
| rs12678919 | LPL |
| rs714052 | MLXIPL |
| rs17216525 | NCAN |
| rs7679 | PLTP |
| rs2954029 | TRIB 1 |
| rs7819412 | XKR6 |
| rs964184 | ZNF259 |

Finally, genotypes at other markers, rs9939609 (FTO), rs5082 (APOA2), rs1800588 (LIPC), rs10850219 (KCTD I 0), rs2241201 (MMAB) are also part of the logic module.

Having received a digitized genome dataset and having calculated the risk as described, a logic module of the rules library is in condition for execution. Under direction of the application code, the logic processor calls a logic module from the rules library. The logic module receives as input particular information from the genome dataset and additionally from the risk assignment calculations.

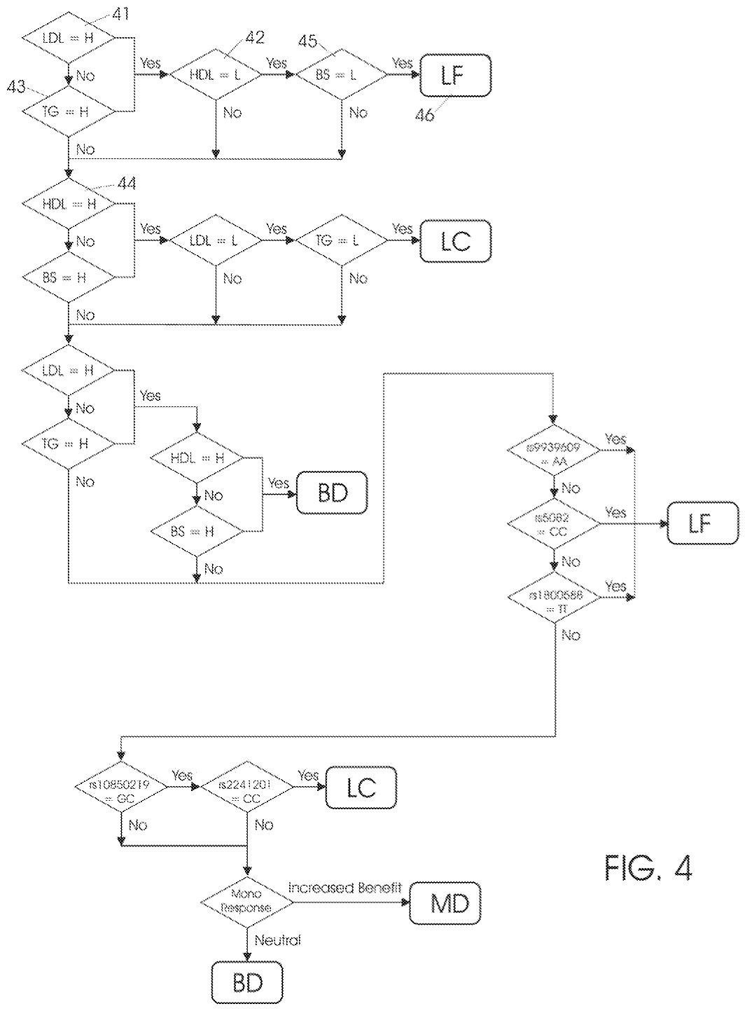

One important example of a logic module of these systems is developed with reference to drawing FIG. 4. The logic module is expressed as a finite set of “If-Then” conditionals with branching as shown below.

| Step | IF | THEN |
|---|---|---|
| 1 | GR = High for LDL OR TG | LF |
| | AND | |
| | GR # High for HDL | |
| | AND | |
| | GR # High for Sugar | |
| 2 | GR = High for HDL OR Sugar | LC |
| | AND | |
| | GR # High for LDL | |
| | AND | |
| | GR # High for TG | |
| 3 | GR = High for LDL OR TG | BD |
| | AND | |
| | GR = High for HDL OR Sugar | |
| 4 | GR * High for LDL AND TG AND HDL AND Sugar | LF |
| | AND | |
| | rs9939609 = AA OR rs5082 = CC OR rs1800588 = TT | |
| 5 | GR * High for LDL AND TG AND HDL AND Sugar | LC |
| | AND | |
| | rs10850219 = GG | |
| | AND | |
| | rs2241201 = CC | |
| 6 | GR # High for LDL AND TG AND HDL AND Sugar | MD |
| | AND | |
| | Response to Monounsaturated Fats** = “Increased | |
| | Benefit” | |
| 7 | None of the above | BD |

In a first execution step 41, a branching conditional asks whether LDL has been determined as ‘high’ for this user's genome. If LDL is ‘high’, then execution continues to the conditional 42. If LDL is ‘low’ then execution continues, to conditional 43. Conditional 43 considers whether there is a high genetic risk of increased triglycerides for this user in view of the genetic dataset and where there is, the execution follows again to conditional 42. If there is neither high genetic risk for increased LDL nor high genetic risk for increased TG then execution passes to conditional 44. However, if either LDL or TG is ‘high’ then HDL is considered in conditional 42 and perhaps, depending upon risk value assigned to HDL and further to conditional 45 where assigned risk value relating to elevated blood sugar BS is considered. If risk of elevated blood sugar is affirmed as Low in conditional 45, the logic module execution ends at logic module endpoint 46 where a low fat diet is specified and recommended to this person.

Of course all experts in logic execution will easily verify that the logic tree set forth always ends in either of a minimum of four diet action plans including low-fat (LF), low carbs (LC), balanced diet (BD), or Mediterranean diet (MD). Where a low fat diet includes a lower calorie diet where the reduction in calories comes from reducing the amount of fat in the diet. A low carb diet includes a lower calorie diet where the reduction in calories comes from reducing the amount of carbs in the diet. A balanced diet includes a lower calorie diet with the goal of keeping a keeping a healthy balance of all macronutrients. While a Mediterranean diet includes a lower calorie diet with the goal of substituting saturated and other fats with monounsaturated fats.

TABLE V

| | Response to | |
|---|---|---|
| | Monounsaturated | |
| Reported Item | Fats | |
| rs1801282 C > G | rs17300539 G > A | |
| (PPARG) | (ADIPOQ) | Outcome |
| CC | AA | Increased Benefit |
| CG | AA | Increased Benefit |
| GG | AA | Increased Benefit |
| CC | GA | Increased Benefit |
| CG | GA | Increased Benefit |
| GG | GA | Increased Benefit |
| CC | GG | Neutral |
| CG | GG | Increased Benefit |
| GG | GG | Increased Benefit |

While it is a goal of the systems taught herein to suggest diet plans to users for optimal health based upon their own genetic ‘signaluret, it is additionally useful to at the same time make other health related suggestions regarding diet where certain genetics features are also found. Therefore, a diet plan 51 of these systems may include several components. Among them a weight control portion 52, an “important nutrients” section 53 and a specific diet related disease section 54.

In some persons it may be found that a specific mutation which exposes the person to an increased risk of a known disease or condition which can be mitigated with certain dietary nutrients. In one example, a person having a genetic predisposition to prostate cancer might be instructed to increase intake of tomatoes in a diet as there is evidence that tomatoes may reduce this risk. Where genetic markers for prostate cancer are found, a diet recommendation might include a weight control portion and in addition a list of dietary guidelines which may reduce risk of prostate cancer and can be used in further cooperation with the weight control portion of the diet action plan.

In another example where information in the genome suggests diet modifications which cooperate with the diet action plan suggested, certain SNPs will indicate a diet related disease such as celiac disease. Celiac disease requires a person's diet to be modified to exclude gluten. Accordingly, any determination of a useful weight control diet type may be further modified to exclude gluten where it is also determined that the user's genome includes markers for celiac disease. As such, these systems also account for artifacts in the genome which have implications other than those which relate to weight control, and where they do, reports may also include accommodation for that.

In review, the broadest versions of these systems are best understood by considering the following description.

An apparatus for health management which is based upon genetics testing is made up of several major components. These major components and the relationships there between in preferred versions of these apparatus are as follows. A genetic scanner is coupled to a logic processor. The genetics scanner is arranged to receive genetic matter (such as DNA or RNA) from a human test subject at an input port of the scanner. The genetic matter is processed by the scanner. In particular, a plurality of optical signals are thresholded to form a binary representation of the test subject's genome. This binary representation or ‘dataset’ is passed to a logic processor for further processing. Stored program code includes analysis modules with conditional branchings which depend upon the element of the genome. Where certain features of the genome are found to be present, the logic flow of the analysis module is switched. After full execution of these—analysis modules, the resulting output is used to drive variable control objects of a report template. Report templates stored in a report engine include many of these control objects which are responsive to the particular outputs of the analysis modules.

An important part of these apparatus is prepared, stored program code. It is not sufficient that a general purpose computer be used—rather, a computer having specialty software installed thereon is required. This specialty software or ‘program code’ is embodied as two distinct portions. In a first portion, program code includes application code. In addition to the application code, program code also includes a rules library having therein at least one logic module which may be executed by the application code. The application code runs to conduct performance of the apparatus as a whole. After a dataset digital genome particular to a specific test subject is received from the genetic scanner, the application code invokes various of the logic modules particular to features and values of the dataset. Upon completion of execution of these modules, the application code provides as output to the report engine parametric values which are coupled to and drive the steady states of various control objects from which these report templates are comprised. The logic modules receive as inputs various features present in the digital representation of the genetic signature. In particular, these logic modules are sometimes arranged to consider a plurality of markers in the genome—where each of those markers relates to a certain disease for example. One important output of such module might be an overall risk assessment for that condition or disease. For example, a binary value representing high risk or low risk of developing a disease might be the output of one of these logic modules. A large group of genetic markers is considered, and then a declaration of high or low is output as a risk assessment particular to the genome under analysis. In more advanced versions, risk assessment is not handled as a binary, but rather a quinary value. Other logic modules which cooperate with preliminary risk assessment modules also are included in the rules library. For example, a logic module may receive as input a risk assessment value associated with a particular disease or health condition. Based upon those risk assessments and in further view of other genetic markers present in the genome, these logic modules also use branching logic to arrive at a discrete output. In one important example, a logic module receives risk assessment values and genetic information as inputs and processes that information to arrive at a diet type specification as output. One will now fully appreciate how an automated genetics based health management systems may be used for selection of appropriate diet and nutritional recommendations. Although the present invention has been described in considerable detail with clear and concise language and with reference to certain preferred versions thereof including best modes anticipated by the inventors, other versions are possible. Therefore, the spirit and scope of the invention should not be limited by the description of the preferred versions contained therein, but rather by the claims appended hereto.

1. A genetics based health management apparatus configured to facilitate improved generation of a genome specific diet type action plan for improved health management and to facilitate behavior modification by modifying dietary actions included in the genome specific diet type action plan to reflect not only weight control diet plans designed for optimal health but also to reflect diet plans that are customized based on artifacts identified within a person's genome, which artifacts have implications other than those related to weight control, said apparatus comprising:
a genetic scanner;
a logic processor;
stored program code executable by said logic processor; and
a report engine that produces a report,
wherein said stored program code comprises application code and a rules library, and said application code is arranged to direct execution of at least one logic module from the rules library in view of digital genomes received from the genetic scanner; and
wherein said genetic scanner is communicatively coupled to said logic processor, and said report engine is communicatively coupled to said logic processor, whereby the report produced by said report engine depends upon results from execution of said program code, wherein said stored program code configures the logic processor to at least:
receive input from the genetic scanner, the input including details about a plurality of genetic markers;
determine, based on presence or absence of a first set of the genetic markers, a first risk factor associated with an LDL level as either above or below a first threshold based on the received input;
determine, based on presence or absence of a second set of the genetic markers, a second risk factor associated with an HDL level as either above or below a second threshold based on the received input;
determine, based on presence or absence of a third set of the genetic markers, a third risk factor associated with a blood sugar (BS) level as either above or below a third threshold based on the received input;
determine, based on presence or absence of a fourth set of the genetic markers, a third risk factor associated with a triglyceride (TG) level as either above or below a third threshold based on the received input;
determine a fourth risk factor associated with one or more of eating behavior, metabolism, blood lipids, blood proteins, metabolites, BMI, and taste; and
generate the report, which includes a customized diet plan designed for optimal health based upon the first risk factor, the second risk factor, the third risk factor, the fourth risk factor, and the fifth risk factor, wherein the report includes a weight control portion, a nutrient section, and a specific diet-related disease section, and wherein artifacts identified from the first set of genetic markers, the second set of the genetic markers, the third set of the genetic markers, and the fourth set of the genetic markers are used to modify the customized diet plan to cause the customized diet plan to not only present weight control dietary options for improving health but also to present certain diet modifications that cooperate with the weight control dietary options, where the certain diet modifications relate to dietary guidelines that are identified as potentially reducing a risk of a potential disease that is identified as having a correlation to the first set of genetic markers, the second set of the genetic markers, the third set of the genetic markers, and/or the fourth set of the genetic markers, wherein:
the report indicates a recommendation of a low fat diet in response to the second, and third risk factors each being below the respective second, and third thresholds and the first and fourth risk factors being above the respective first and fourth thresholds;
the report indicates a recommendation of low carbohydrate diet in response to the first and third risk factors being below the respective first and third thresholds, and either the second or fourth risk factors being above the respective second and fourth thresholds; and
the report indicates a recommendation of a balanced diet in response to the first and fourth risk factors being above the respective first and fourth thresholds, and either the second or third risk factors being above the respective second and third thresholds.

2. The genetic based health management apparatus of claim 1, wherein said genetic scanner comprises:
an input port arranged to receive therein genetic material from a human individual;
a threshold facility arranged to analyze analog optical signals to form dataset digital representation of genetic material received at said input port; and
an output port communicatively coupled to said logic processor whereby the dataset digital representation may be conveyed from the genetic scanner to the logic processor.

3. The genetic based health management apparatus of claim 1, wherein said report engine further comprises prescribed report templates, and wherein said report templates comprise a plurality of control objects each having a plurality of states, and the control objects are responsive to said program code whereby they may be set into a current state to reflect values from the logic processor.

4. The genetic based health management apparatus of claim 3, further comprising a document server that is communicatively coupled to remote stations and is arranged to provide executed document templates to authenticated requesting parties as user specific genetic reports.

5. The genetic based health management apparatus of claim 1, further comprising a logic module comprising a risk assignment portion.

6. The genetic based health management apparatus of claim 5, wherein the risk assignment portion comprises a numeric system characterized as any from the group: binary, tertiary, or quaternary or quinary or another numeric system.

7. The genetic based health management apparatus of claim 5, said logic module further comprises a logic portion dependent upon calculated risk values.

8. The genetic based health management apparatus of claim 1, wherein the results are based further on one or more of:
other markers associated with diet, metabolism, behavior or exercise performance responses or other weight/nutrition control topics;
family history, medical history, or lifestyle choices that are collected from a survey; and
other markers associated with disease risk, eating behaviors, taste preference, or food reactions.

9. The genetics based health management apparatus of claim 1, wherein said program code further configures the logic processor to:
determine a first genotype at marker rs9939609 based on the input;
determine a second genotype at marker rs5082 based on the input;
determine a third genotype at marker rs1800588 based on the input; and
generate a report indicating a recommendation of a low fat diet in response to at least the first genotype being “AA”, second genotype being equivalent to “CC”, and the third genotype being equivalent to “TT.”

10. The genetics based health management apparatus of claim 9, wherein said program code further configures the logic processor to:
determine a fourth genotype at marker rs10850219 based on the input;
determine a fifth genotype at marker rs2241201 based on the input; and
generate a report indicating a recommendation of a low fat diet in response to at least the fourth genotype being “GC”, fifth genotype being equivalent to “CC”, and the third genotype being equivalent to “TT.”

11. The genetics based health management apparatus of claim 9, wherein said program code further configures the logic processor to:
determine a fourth genotype at marker rs10850219;
determine a fifth genotype at marker rs2241201; and
generate a report indicating a recommendation of a low carbohydrate diet in response to at least the fourth genotype being “GC”, fifth genotype being equivalent to “CC” and the third genotype being equivalent to “TT.”

12. The genetics based health management apparatus of claim 9, wherein said program code further configures the logic processor to:
quantify a response to monounsaturated fats as either above or below a fifth threshold based on the input; and
generate a report indicating a recommendation of a Mediterranean diet in response to the response to monounsaturated fats being above the fifth threshold.

13. The genetics based health management apparatus of claim 1, wherein said program code further configures the logic processor to:
determine a first SNP marker for rs1883025 based on the input;
quantify the second risk factor based on whether the first SNP marker is equivalent to “ABCA1.”

14. A genetic based health management method for facilitating improved generation of a genome specific diet type action plan for improved health management and to facilitate behavior modification by modifying dietary actions included in the genome specific diet type action plan to reflect not only weight control diet plans designed for optimal health but also to reflect diet plans that are customized based on artifacts identified within a person's genome, which artifacts have implications other than those related to weight control, comprising:
receiving genetic material from a human individual;
reacting said genetic matter with a set of prescribed gene probes;
scanning said reactions to form a digitalized genome dataset, the dataset indicating a presence or absence of a plurality of genetic markers;
determining, via a logic processor, a first risk factor associated with LDL levels as either above or below a first threshold based on a presence or absence of a first set of the genetic markers from the digitalized genome dataset;
determining, via a logic processor, a second risk factor associated with HDL level as either above or below a second threshold based on a presence or absence of a second set of the genetic markers from the digitalized genome dataset;
determining, via a logic processor, a third risk factor associated with blood sugar (BS) level as either above or below a third threshold based on a presence or absence of a third set of the genetic markers from the digitalized genome dataset;
determining, via a logic processor, a fourth risk factor associated with triglyceride (TG) level as either above or below a fourth threshold based on a presence or absence of a fourth set of the genetic markers from the digitalized genome dataset;
determining, via a logic processor, a fifth risk factor associated with one or more of eating behavior, metabolism, blood lipids, blood proteins, metabolites, BMI, and taste; and
generating a report, which includes a customized diet plan designed for optimal health based upon the first risk factor, the second risk factor, the third risk factor, the fourth risk factor, and the fifth risk factor, wherein the report includes a weight control portion, a nutrient section, and a specific diet-related disease section, and wherein artifacts identified from the first set of genetic markers, the second set of the genetic markers, the third set of the genetic markers, and the fourth set of the genetic markers are used to modify the customized diet plan to cause the customized diet plan to not only present weight control dietary options for improving health but also to present certain diet modifications that cooperate with the weight control dietary options, where the certain diet modifications relate to dietary guidelines that are identified as potentially reducing a risk of a potential disease that is identified as having a correlation to the first set of genetic markers, the second set of the genetic markers, the third set of the genetic markers, and/or the fourth set of the genetic markers, wherein the report indicates a recommendation of:
a low fat diet in response to the second, and third risk factors each being below the respective second, and third thresholds and the first and fourth risk factors being above the respective first and fourth thresholds;
a low carbohydrate diet in response to the first and third risk factors being below the respective first and third thresholds, and either the second or fourth risk factors being above the respective second and fourth thresholds; and/or
a balanced diet in response to the first and fourth risk factors being above the respective first and fourth thresholds, and either the second or third risk factors being above the respective second and third thresholds.

15. The genetic based health management method of claim 17, wherein the fifth risk factor is associated with two or more of seating behavior, metabolism, blood lipids, blood proteins, metabolites, BMI, and taste.

16. The genetic based health management method of claim 17, wherein said diet action plan includes one or more diets characterized as: low-fat, low-carb, balanced, and Mediterranean.
